(19) United States
(12) Patent Application Publication
(10) Pub. No.: US 2026/0151079 A1
(43) Pub. Date: Jun. 4, 2026

(54) PREDICTING AWAKE-TIME ALERTNESS

(71) Applicant: Polar Electro Oy, Kempele (FI)

(72) Inventors: Topi KORHONEN, Kempele (FI); Riikka AHOLA, Kempele (FI); Matti Luomala, Kempele (FI); Kaisu Martinmäki, Kempele (FI); Lotta Rönnberg, Kempele (FI); Eve Vallström, Kempele (FI)

(73) Assignee: Polar Electro Oy, Kempele (FI)

(21) Appl. No.: 19/125,331

(22) PCT Filed: Nov. 2, 2023
(86) PCT No.: PCT/FI2023/050611

§ 371 Date: Apr. 29, 2025

(30) Foreign Application Priority Data
Nov. 4, 2022 (GB) 2216409.9

(51) Int. Cl. A61B 5/00; A61B 5/0205; A61B 5/024; A61B 5/11

(52) U.S. Cl. CPC A61B 5/4809; A61B 5/0205; A61B 5/024; A61B 5/1118; A61B 5/4812; A61B 5/4815; A61B 5/681; A61B 5/7275

(57) ABSTRACT

A computer-implemented method for estimating awake-time alertness of a user includes: performing a recursive alertness prediction procedure by performing operations including: obtaining measurement data measured, from a user, by at least one sensor device; determining at least a sleep time, a sleep quality, and a circadian phase of the user; selecting, in response to a preceding awake-time alertness pattern enabled for usage in the recursive alertness prediction procedure, the preceding awake-time alertness pattern as an alertness pattern template; selecting, otherwise, a default alertness model as the alertness pattern template; determining an awake-time alertness pattern; and determining whether to enable or disable the awake-time alertness pattern determined for a subsequent recursive alertness prediction procedure; and causing displaying, to the user via a user interface, the awake-time alertness pattern determined.

### CROSS-REFERENCE TO RELATED APPLICATIONS

This application is a national phase application of International Application No. PCT/FI2023/050611, filed Nov. 2, 2023, which claims benefit and priority to Great Britain Application No. 2216409.9, filed Nov. 4, 2022, which are incorporated by reference herein in their entireties.

### BACKGROUND

### Technical Field

The invention relates to a field of measuring a human and, in particular, to predicting impact of sleep on daytime alertness.

### SUMMARY

Modern activity monitoring devices, sometimes called activity trackers, employ motion sensors to measure user's motion during the day. Some activity monitoring devices may employ other sensors such as physiological or biometric sensors such as heart activity sensors or skin temperature sensors. Some activity monitoring devices are also capable of estimating a sleep time and/or sleep quality of the user.

Sleep time and sleep quality have been shown to have significant effect on awake-time alertness. Therefore, it would be advantageous to provide a method for predicting awake-time alertness such that it reflects the effect of sleeping habits accurately.

According to an aspect, there is provided the subject matter of the independent claims. Embodiments are defined in the dependent claims.

### BRIEF DESCRIPTION OF THE DRAWINGS

In the following, various example embodiments will be described in greater detail with reference to the accompanying drawings, in which

FIG. 1 illustrates a simplified architecture of a system;

FIGS. 2 to 10 are flow charts illustrating example functionalities;

FIGS. 11 and 12 illustrate display views according to some embodiments; and

FIG. 13 illustrates an exemplary embodiment of an apparatus.

### DETAILED DESCRIPTION

The following embodiments are exemplary. Although the specification may refer to “an”, “one”, or “some” embodiment(s) in several locations, this does not necessarily mean that each such reference is to the same embodiment(s), or that the feature only applies to a single embodiment. Single features of different embodiments may also be combined to provide other embodiments. Furthermore, words “comprising” and “including” should be understood as not limiting the described embodiments/examples to consist of only those features that have been mentioned and such embodiments may contain also features/structures that have not been specifically mentioned. Further, although terms including ordinal numbers, such as “first”, “second”, etc., may be used for describing various elements, the structural elements are not restricted by the terms. The terms are used merely for the purpose of distinguishing an element from other elements. For example, a first element could be termed a second element, and similarly, a second element could be also termed a first element without departing from the scope of the present disclosure.

Different embodiments and examples are described below using single units, models, equipment, and memory, without restricting the embodiments/examples to such a solution. Concepts called cloud computing and/or virtualization may be used. The virtualization may allow a single physical computing device to host one or more instances of virtual machines that appear and operate as independent computing devices, so that a single physical computing device can create, maintain, delete, or otherwise manage virtual machines in a dynamic manner. It is also possible that device operations will be distributed among a plurality of servers, nodes, devices, or hosts. In cloud computing network devices, computing devices and/or storage devices provide shared resources. Some other technology advancements, such as Software-Defined Networking (SDN), may cause one or more of the functionalities described below to be migrated to any corresponding abstraction or apparatus or device. Correspondingly, Web 3.0, also known as the third-generation internet, implementing for example blockchain technology, may cause one or more of the functionalities described below to be distributed across a plurality of apparatuses or devices. Therefore, all words and expressions should be interpreted broadly, and they are intended to illustrate, not to restrict, the embodiment.

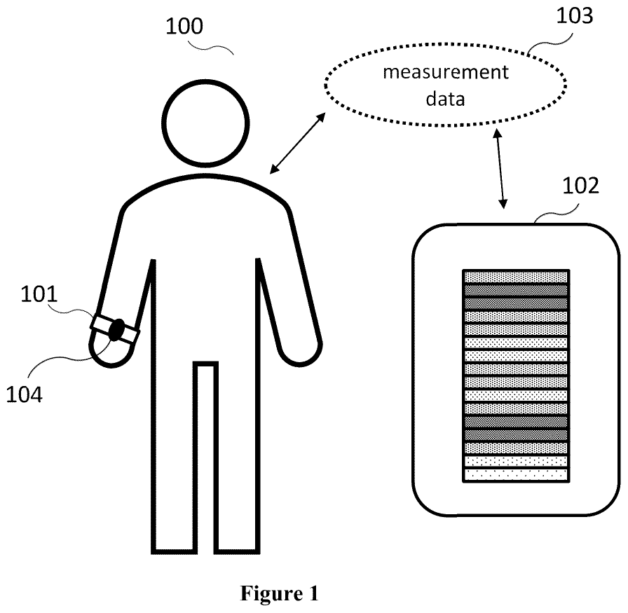

A general exemplary architecture of a system estimating awake-time alertness of a user is illustrated in FIG. 1. FIG. 1 is a simplified system architecture showing only some devices, apparatuses, and functional entities, all being logical units whose implementation and/or number may differ from what is shown. The connections shown in FIG. 1 are logical connections; the actual physical connections may be different. It is apparent to a person skilled in the art that the system comprises any number of shown elements, other equipment, other functions, and other structures that are not illustrated. They, as well as the protocols used, are well known by persons skilled in the art and are irrelevant to the actual invention. Therefore, they need not to be discussed in more detail here.

In the example illustrated in FIG. 1, the system 100 comprises at least a processing circuitry configured to analyse measurement data 103 measured from a user, for example by carrying out methods described in more detail below. The processing circuitry may be realised in a wearable device 101 worn by the user, such as a smart watch or a wrist-worn wearable training computer or an activity tracker device. The processing circuitry may be realised in a user device 102 such as a smart phone or a tablet computer. The processing circuitry may be realised in a server computer such as a cloud server. The measurement data 103 may be provided by at least one sensor device 104 which may be comprised in the wearable device 101 or in the user device 102, or the sensor device may be external to the wearable device 101 or the user device 102 but provided with data transfer capability with the wearable device 101 or the user device 102, respectively. In some embodiments, the sensor device is provided with the data transfer capability with the server computer via one or more gateway devices routing the connection be-tween the sensor device and the server computer via one or more computer networks or communication networks. The wearable device 101, the user device 102, the server computer, and/or the sensor device 104 may be connectable over one or more networks, over a short-range wireless connection such as Bluetooth, or over a Universal Serial Bus (USB) connection.

The sensor device 104 may measure one or more of the following features from the user: motion, electrocardiogram (ECG), photoplethysmogram (PPG), electroencephalography (EEG), bioimpedance, galvanic skin response, body temperature, respiration, electrooculogprahy (EOG), or ballistocardiogram (BCG). For measuring motion, the sensor device 104 may comprise an inertial sensor such as an accelerometer, a gyroscope, or a magnetometer, or any sensor fusion that is any combination of these motion sensors, and the sensor device 104 may output motion measurement data. The sensor device 104 measuring ECG, PPG, or BCG may output heart activity measurement data 103. The sensor device 104 may comprise one or more electrodes attachable to the user's skin to measure an electric property from the skin which, through appropriate signal processing techniques, may be processed into an ECG signal. In some techniques, the heart activity measurement data 103 may represent appearance of R waves of electric heart impulses. In PPG measurements a light emitted by a light emitter diode or a similar light source and reflected back from the user's skin is be sensed by using a photo diode or a similar light sensing component. The sensed light is then converted into an electric measurement signal in the light sensing component and signal processing is used to detect desired signal components from the electric measurement signal. In the PPG measurements, P waves may be detected which enables computation of a PP interval and a heart rate, for example. The sensor device 104 measuring the EOG may output electric measurement data 103 representing eye motion. The sensor device 104 may comprise a special-purpose respiration sensor outputting a respiratory rate. The respiratory rate may also be measured from the heart activity measurements.

The user device 102 refers to a computing device (equipment, apparatus) and it may also be referred to as a user terminal, a user apparatus, mobile device, or a mobile terminal. Portable computing devices (apparatuses) include wireless mobile communication devices operating with or without a subscriber identification module (SIM) in hardware or in software, including, but not limited to, the following types of devices: mobile phone, smartphone, personal digital assistant (PDA), handset, laptop and/or touch screen computer, tablet (tablet computer), multimedia device, wearable computer, such as smart watch, and other types of wearable devices, such as clothing and accessories incorporating computer and advanced electronic technologies. The user device 102 may comprise one or more user interfaces. The one or more user interfaces may be any kind of a user interface, for example a screen, a keypad, a loudspeaker, a microphone, a touch user interface, an integrated display device, and/or external display device.

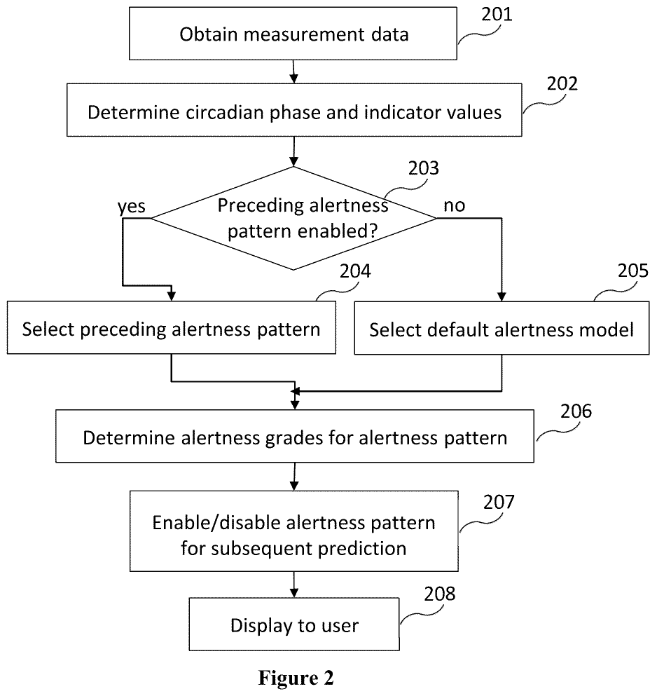

According to the exemplary embodiment illustrated in FIG. 2 a recursive awake-time alertness prediction procedure is performed by the processing circuitry, for example in the wearable device 101 or the user device 102.

Referring to FIG. 2, measurement data measured from the user by at least one sensor device is obtained in block 201. The measurement data obtained may comprise various measurement data types as explained earlier with FIG. 1. The measurement data may be obtained from a memory or a data storage of the sensor device, the wearable device, the user device, and/or the server computer. The measurement data may originate from the sensor device and may have been streamed from the sensor device over at least one wired or wireless interface.

A circadian phase of the user is determined in block 202 using the measurement data obtained. The circadian phase may be understood as reflecting a timing of different states of a circadian rhythm of a human during a (calendar) day. The circadian rhythm (equivalently circadian process, circadian cycle) may be understood as an internal physiological process regulating a natural sleep-wake rhythm of the user. The circadian rhythm repeats approximately every 24 hours. If the user follows the natural sleep-wake rhythm regulated by the suprachiasmatic nucleus (SCN) of the anterior hypothalamus (the circadian system), the natural sleep-wake rhythm would substantially follow the circadian rhythm. However, people often deviate from the natural sleep-wake rhythm because of social activities, personal preferences to stay awake during weekends, travelling across time zones, shift work, etc. As a consequence, the sleep-wake rhythm of the user may either follow the circadian rhythm or deviate from it, and this also affects the alertness of the user. This results from the fact that the sleep-wake rhythm affects the sleep propensity during the awake-time via sleep homeostasis known in the literature. Misalignment between the sleep-wake rhythm and the circadian rhythm may degrade alertness in at least some parts of the following day(s), depending on the degree of the misalignment. The sleep-wake rhythm follows the user's actions of going to sleep and waking up, while the circadian rhythm follows the sleep-wake rhythm at a slower pace. The sleep-wake rhythm may be measured by using state-of-the-art sleep analysis sensors and metrics. There exists a vast amount of literature and commercially available products for measuring the sleep start and sleep end times that define the sleep-wake rhythm and, therefore, detailed description thereof is omitted. For example, the sleep analysis functions used in Polar Vantage V or Polar Grit X may be employed to determine the sleep-wake analysis. The sleep start time and the sleep end time may be measured by using the above-described heart activity sensor and/or a motion sensor.

Additionally, one or more indicator values related to the user's circadian rhythm and/or sleep-wake rhythm are determined in block 202, comprising at least a sleep time and a sleep quality. The sleep time indicates how long the user has been asleep during the measuring of the measurements. The sleep quality may be understood as a metric indicating how restorative a sleep event is. The exact estimation of the sleep time and sleep quality is beyond the scope of this description, and the method disclosed in U.S. Pat. No. 10,993,656 by Polar Electro may be employed.

Referring to FIG. 2, it is resolved in block 203 whether a preceding awake-time alertness pattern (a first awake-time alertness pattern) is enabled for usage in the recursive alertness prediction procedure. The preceding alertness pattern comprises a plurality of time intervals and an alertness grade per time interval. The alertness grades may be, for example, selected amongst a pre-determined plurality of alertness grades or determined from a pre-determined alertness grade range. If the preceding alertness pattern is enabled (block 203: yes), the preceding alertness pattern is selected in block 204 as an alertness pattern template. If the preceding alertness pattern is not enabled (block 203: no), a default alertness model is selected in block 205 as the alertness pattern template. The default alertness model comprises the plurality on time intervals and an alertness grade per time interval. The alertness grades may be, for example, selected amongst the pre-determined plurality of alertness grades or determined from the pre-determined alertness grade range. Awake-time alertness pattern (a second awake-time alertness pattern) is determined in block 206 by determining a plurality of alertness grades for the plurality of time intervals, an alertness grade per time interval, using the sleep time, the sleep quality, the circadian phase, and the alertness pattern template selected. It is then determined in block 207 whether to enable or disable the determined awake-time alertness pattern for a subsequent alertness prediction procedure. Displaying the awake-time alertness pattern is caused in block 208 to the user via a user interface, for example of the user device. The alertness grades in the awake-time alertness pattern may be displayed in the form of score values within a determined range, e.g. 1 to 10, 0 to 10, or 0 to 100. The alertness grades may be provided in a verbal form, e.g. “High”, “Compromised”, and “Poor”, or “Good”, “Fair”, and “Modest”. The alertness grades may be provided in a coloured form, e.g. blue for high alertness, turquoise for compromised alertness, and green for poor alertness, or darker shades for high alertness and lighter shades for poor alertness. However, these are just examples of indicating the alertness pattern, and numerous other display techniques may be utilised.

The determined awake-time alertness pattern may be used for detecting a lowered alertness level of the user, and an alertness warning of the lowered alertness level may be given to the user via the user interface of the user device or the wearable device. In an embodiment if the awake-time alertness pattern does not include an alertness grade indicating a highest alertness level, the alertness warning is given. In another embodiment, an average of the alertness grades in the alertness pattern is determined. If the average of the alertness grades is below a pre-determined average alertness threshold, the alertness warning is given to the user, and otherwise no warning is given. In yet another embodiment the warning or alarm is dynamically based on a current alertness grade, that is, an alertness grade determined for a current time interval. If the current alertness grade is below a pre-determined alertness threshold, the warning is given to the user, and otherwise no warning is given. The lowered alertness level may be detected also by using a combination of the rules described here. The user may set his/her preferences for receiving alertness warnings via the user interface. The alertness warning may be given, for example, as a sound or buzz alarm or a visual indication.

As described above, the alertness is based on the circadian phase and the sleep homeostasis. Both of these develop at such a slow pace that the development spans over multiple days. For example, a severe sleep debt takes more than one night's good sleep to recover. Similarly, a substantial change in the sleep-wake rhythm, e.g. travelling across time zones or changing from a morning shift to a night shift work, shifts the circadian phase over the following days. Therefore, it is advantageous to make the estimation of the daily awake-time alertness pattern a recursive procedure to reflect the slow changes in the sleep homeostasis and the circadian phase. However, the measurement data may be corrupted for some reasons and, therefore, the effect of the corrupted data should be limited so that it does not corrupt the daily awake-time alertness patterns for several following days. One reason for the corrupted data may be poor skin contact of a skin temperature sensor used for measuring the skin temperature and for subsequent estimation of the circadian phase. Another reason may be the user omitting to wear the sensor device 104 during the primary sleep. Therefore, validation of the daily awake-time alertness pattern and related enablement or disablement of the awake-time alertness pattern for the subsequent iteration improves the reliability of the recursive awake-time alertness pattern estimation.

In an embodiment of the alertness prediction procedure the measurement data obtained is converted from a local time of the user into a universal time frame, e.g. the coordinated universal time (UTC). The awake-time alertness pattern is determined in the universal time frame, and it is then converted to the local time of the user. An advantage of this embodiment is that the travel across the time zones will not generate gaps or overlaps in the measurement data time stamps. The conversion may be realized by time-stamping the measurement data in the UTC clock in which case the local time-reference is converted from the local time to the UTC for the purpose of time-stamping the measurement data. Another option would be to time-stamp the measurement data in the local time and perform the time conversion to the UTC for the purpose of determining the awake-time alertness pattern in the UTC. In both cases, the awake-time alertness pattern is displayed to the user in the local time.

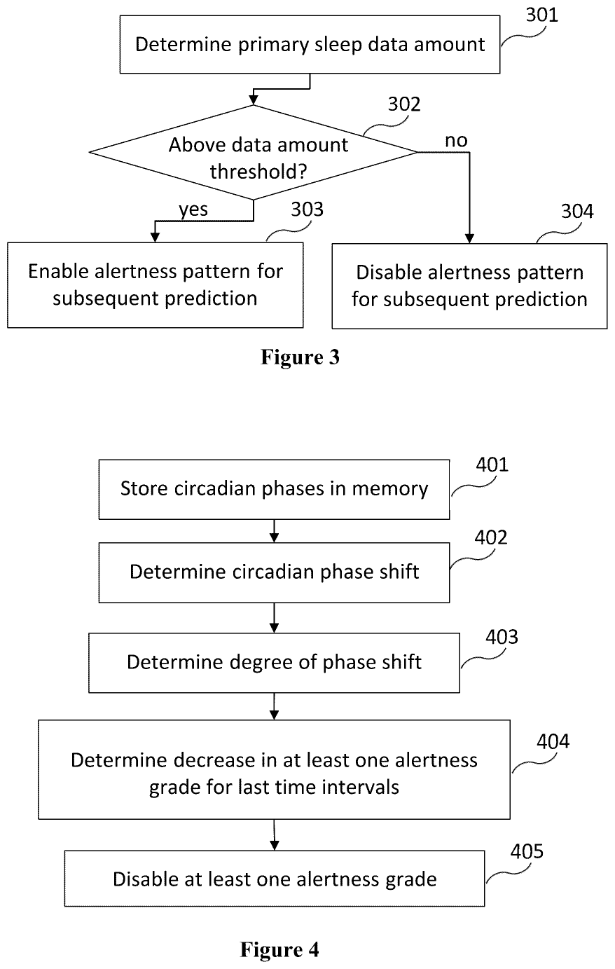

According to the exemplary embodiment of FIG. 3 the determining whether to enable or disable the alertness pattern for a subsequent alertness prediction procedure is performed using an amount of a primary sleep detected from the measurement data. As described in connection with some embodiments herein, the user may forget to wear the sensor device or the skin contact may be poor, resulting in that the sleep cannot be measured at all or there are gaps in the measurement data. Such lack of measurement data may invalidate the awake-time alertness pattern estimation, and it may be feasible to disable the recursion of such an invalid awake-time alertness pattern estimation. There are several embodiments for realizing the disablement. Let us assume in this case that it takes seven days for the recursive alertness model to converge to the user's sleeping habits. If from the past seven primary sleep events there is measurement data for all primary sleep events, the awake-time alertness pattern may be enabled for the subsequent recursion. If there is more than zero but less than a first number (e.g. one or two) of primary sleep events having no measurement data (optionally having the artificially generated measurement data as described below), the awake-time alertness pattern may still be enabled for the subsequent recursion. The user may be indicated that the awake-time alertness pattern accuracy may be degraded. If there is more than the first number of primary sleep events having no measurement data (optionally having the artificially generated measurement data as described below), the awake-time alertness pattern may be disabled for the subsequent recursion(s) and, instead, the recursion may start from the default template.

With respect to the definition of the primary sleep, the primary sleep refers to the sleep that the user takes daily and that is usually 6 to 10 hours long, depending on the person. The user typically enters various sleep stages including deep sleep and REM sleep during the primary sleep, in multiple sleep cycles. A shortened primary sleep causes sleep debt and extra sleepiness during awake-time, reducing alertness. The functionalities illustrated in FIG. 3 may be performed within block 207 in FIG. 2.

Referring to FIG. 3, an amount of primary sleep measurement data comprised in the measurement data obtained is determined in block 301. It is resolved in block 302 whether the amount of primary sleep measurement data is above a pre-determined data amount threshold. The data amount threshold may define a minimum threshold from which a reliable sleep analysis can be made for the purpose of awake-time-alertness estimation. The minimum threshold may define, for example, that there shall be at least 80 per cent (%) of the maximum amount of measurement data available for the primary sleep. If the sleep duration has been eight hours, there shall then be measurement data for at least 6.4 hours, meaning that the skin contact may be lacking for at most 1.6 hours in order to still qualify the measurement data and resulting awake-time alertness pattern for the subsequent recursion. 80% is just an example, and the threshold may define another amount, e.g. 85% or 90%. If the amount of measurement data for the primary sleep is below the threshold (block 303: no), the awake-time alertness pattern may be estimated but its recursion disabled so that it will not be used as the template for the following recursion (block 304). On the other hand, if it is determined that the amount of measurement data is sufficient (above the threshold, block 303: yes), the awake-time alertness pattern is enabled for the subsequent recursion (block 303). In this manner, invalid measurement data will not corrupt the awake-time alertness patterns of the subsequent days and yet the awake-time alertness pattern is computed even in a case of lacking or invalid measurement data to help the user to evaluate his/her awake-time alertness.

In an embodiment, a difference between the sleep time and a pre-determined target sleep time may be determined. The target sleep time may be preset by the user as a user input, or it may be determined using information received as a user input, such as, for example, age or activity and preset for the user. The target sleep time may also be determined using the sleep-wake rhythm determined over a longer period of time, e.g. over one or more weeks. In an embodiment, the target sleep time is based on the user input and, if the target sleep time received as the user input is outside a predefined target sleep range, the target sleep time may be scaled or shifted to be within the predefined target sleep range. The predefined target sleep range may be based on general sleep science or on the user's sleep history and the range of sleep times of those primary sleep events when the user has slept with a sleep quality above a determined sleep quality threshold. The target sleep range may be six to nine hours, for example. For example, the user may input ten hours as his/her target sleep time. If the sleep history or the sleep science shows that such a long primary sleep is not suitable for the user, the target sleep time may be shifted to the upper end of the target sleep range. As another example, the user may input five hours as the target sleep time. However, it is generally known that such a short sleep time is not healthy and, in such a case, the target sleep time may be shifted to the lower end of the target sleep range. In general, the user-input target sleep time may be shifted to the closest end of the target sleep range, if the user-input target sleep time is outside the target sleep range.

If the difference between the target sleep time and the sleep time of the user is above a pre-determined sleep difference threshold, the alertness of the user may suffer from a sleep debt. Therefore, at least one alertness grade is disabled for the awake-time alertness pattern and for the subsequent alertness prediction procedure, wherein the at least one alertness grade disabled comprises an alertness grade indicating the highest alertness in a range of alertness grades for the awake-time alertness pattern. If the at least one alertness grade is disabled, alertness grades indicating lower alertness(es) in the range of alertness grades may be used instead for the awake-time alertness pattern. The memory may store rules for disabling the maximum alertness grade(s) as a function of the sleep debt. The greater the sleep debt, the greater number of the highest alertness grades may be disabled, as defined in the rules. Accordingly, the apparatus performing the method of FIG. 2 or any one of its embodiments may utilize the rules for enabling and disabling the alertness grades for the awake-time alertness pattern determination of block 206 on the basis of the sleep debt estimated from the measurement data. The gist behind this is that a significant sleep debt causes a dramatic decrease in the awake-time alertness of the following day, irrespective of the awake-time alertness of the previous recursions. By disabling at least some of the highest alertness grades facilitates reflecting this characteristic in the awake-time alertness pattern, thus improving its accuracy.

FIG. 4 illustrates an exemplary embodiment of the alertness prediction procedure taking account of a circadian phase shift. The functionalities illustrated in FIG. 4 may be carried out within block 206 in FIG. 2.

Referring to FIG. 4, the circadian phases determined using the measurement data obtained are stored in block 401 in a memory of, for example, the server computer, the wearable device, or the user device. A phase shift in at least one circadian phase with respect to a preceding circadian phase is determined in block 402. A degree of the phase shift is determined in block 403. A decrease in alertness grades of at least a subset of the plurality of time intervals of the awake/time alertness pattern is determined in block 404. The decrease is in direct proportion to the degree of the phase shift. The subset of the plurality of time intervals comprises at least some of the last adjacent time intervals of the awake-time alertness pattern. At least one alertness grade for the awake-time alertness pattern is disabled in block 405 for the subset of the plurality of time intervals. The at least one alertness grade disabled comprises an alertness grade indicating the highest alertness for the subset of the plurality of time intervals of the awake-time alertness pattern. The gist behind this embodiment is that whenever the user changes his/her daily sleep-wake rhythm and causes a change in the circadian rhythm, the alertness degrades but only for the last time intervals of the awake-time (the later evening), contrary to the effect of the sleep debt that degrades the alertness of the whole awake-time. The number of last time intervals of reduced alertness (disabled alertness grades) and the number of disabled highest alertness grades may be proportional to the degree of the phase shift. The memory may store rules for disabling the maximum alertness grade(s) and for the different subsets of time intervals as a function of the circadian phase shift. The greater the circadian phase shift, the greater number of the highest alertness grades may be disabled and for the greater number of last time intervals of the awake-time, as defined in the rules. Accordingly, the apparatus performing the method of FIG. 2 or any one of its embodiments may utilize the rules for enabling and disabling the alertness grades for the selected number of last time intervals of the awake-time alertness pattern determined in block 206, on the basis of the circadian phase shift determined from the measurement data. By disabling at least some of the highest alertness grades facilitates reflecting the effect of changing the circadian phase in the awake-time alertness pattern, thus improving its accuracy and enabling the user to see the effects of shift work, travelling across time zones etc.

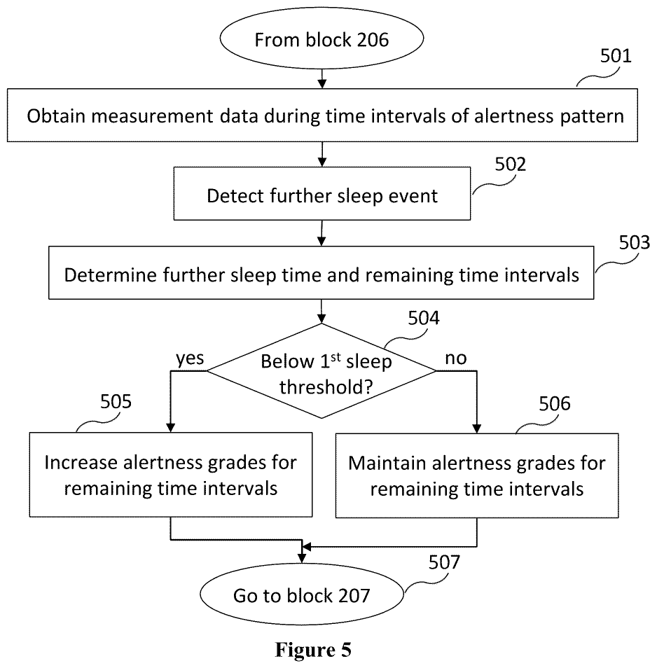

FIG. 5 illustrates an exemplary embodiment of the alertness prediction procedure taking account of a further sleep event. A further sleep, or a secondary sleep refers to naps taken between the primary sleep events, and a nap lasts typically from 10 minutes to a couple of hours. The user conventionally does not enter the deep sleep stage during the secondary sleep, unless the nap is exception-ally long and the user very tired. One factor affecting the alertness is whether or not the user has taken a nap (secondary sleep) during the daytime and the length of the nap. If the nap has been short (less than a sleep amount threshold, e.g. less than 30 minutes), the alertness may be increased. On the other hand, if the nap has been long, e.g. one or two hours, there is a great potential that the user's sleep homeostasis has changed so that the alertness during the remaining daytime is lower than that indicated by the primary sleep time and sleep quality. The alertness degradation may result from sleep inertia of such a long nap. Furthermore, the long nap may cause the user to enter the primary sleep later and, thus, change the circadian phase of the user and reduce the awake-time alertness of the following day. The functionalities illustrated in FIG. 5 may be carried out between blocks 206 and 207 in FIG. 2.

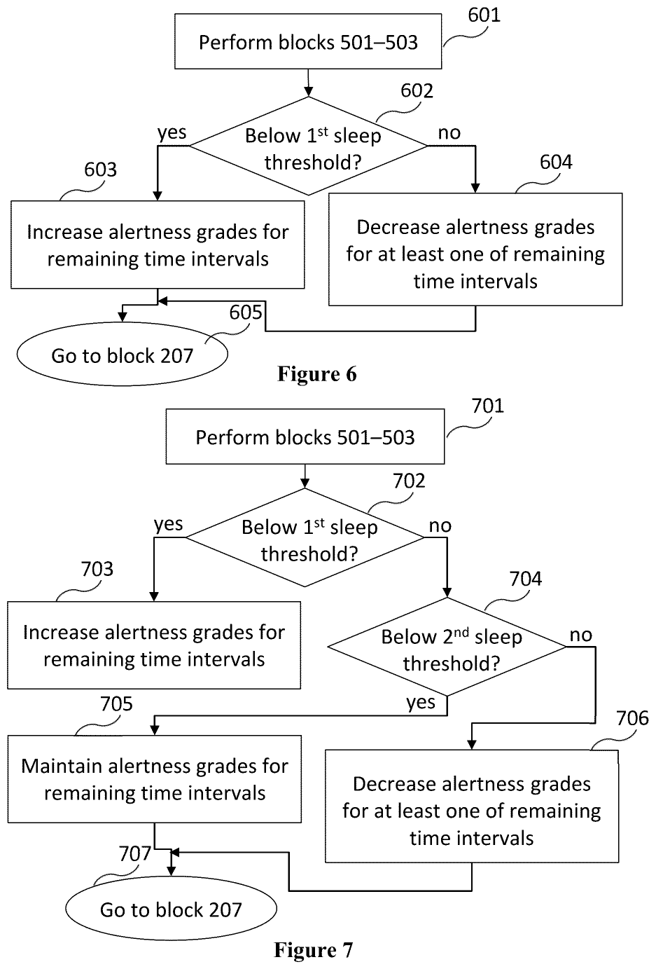

Referring to FIG. 5, measurement data measured from the user during at least one time interval of the awake-time pattern is obtained in block 501. A further sleep event of the user is detected in block 502 in the measurement data measured during the at least one time interval of the awake-time alertness pattern. A further sleep time is determined in block 503 using the measurement data measured during the at least one time interval of the awake-time alertness pattern. The measurement data may include at least one of heart activity measurement data, motion measurement data, and skin temperature measurement data, or any combination of these. Also remaining time intervals of the awake-time alertness pattern are determined in block 503. It is resolved in block 404 whether the further sleep time is below a pre-determined first sleep threshold. The first sleep threshold may be determined based on a sleep history of the user and/or a plurality of preceding awake-time alertness patterns or it may be a pre-determined fixed value. For example, if the sleep homeostasis indicates sleep debt, a greater first sleep threshold may be configured than in a case where there is no sleep debt. If the further sleep time is below the first sleep threshold (block 504: yes), the alertness grades for the remaining time intervals in the awake-time alertness pattern determined are increased in block 505. The increase may be, for example, a constant increase in the alertness grades or it may be proportional to how long time period there is between the end of the further sleep event and the respective time interval. This reflects the alertness boost gained with so-called power naps. If the further sleep time is above the first sleep threshold (block 504: no), the alertness grades for the remaining time intervals in the awake-time alertness pattern are maintained in block 506. A prolonged nap may negate the positive effects on the alertness. The process is continued in block 507 to block 207 in FIG. 2. FIG. 6 illustrates an exemplary embodiment with a possibility to decrease alertness grades due to a longer further sleep time.

Referring to FIG. 6, the further sleep time is determined in block 601 by performing, for example, the functionalities described above with blocks 501 to 503 of FIG. 5. It is resolved in block 602 whether the further sleep time is below the pre-determined first sleep threshold. If the further sleep time is below the first sleep threshold (block 602: yes), the alertness grades for the remaining time intervals in the awake-time alertness pattern determined are increased in block 603. The increase may be, for example, a constant increase in the alertness grades or it may be proportional to how long time period there is between the end of the further sleep event and the respective time interval. If the further sleep time is above the first sleep threshold (block 602: no), the alertness grades for at least one of the remaining time intervals in the awake-time alertness pattern are decreased in block 604. This reflects the adverse effects of the prolonged secondary sleep and resulting sleep inertia on the alertness for the remaining awake time. The decrease may be, for example, a constant decrease in the alertness grades or it may be proportional to how long time period there is between the end of the further sleep event and the respective time interval. The process is then continued in block 605 to block 207 in FIG. 2.

FIG. 7 illustrates an exemplary embodiment with a second sleep threshold that is higher than the first sleep threshold.

Referring to FIG. 7, further sleep time is determined in block 701 by performing, for example, the functionalities described above with blocks 501 to 503 of FIG. 5. It is resolved in block 702 whether the further sleep time is below the pre-determined first sleep threshold. If the further sleep time is below the first sleep threshold (block 702: yes), the alertness grades for the remaining time intervals in the awake-time alertness pattern determined are increased in block 703. The increase may be, for example, a constant increase in the alertness grades or it may be proportional to how long time period there is between the end of the further sleep event and the respective time interval. If the further sleep time is above the first sleep threshold (block 702: no), it is then resolved in block 704 whether the further sleep time is below a pre-determined second sleep threshold. The second sleep threshold may be determined based on a sleep history of the user and/or a plurality of preceding awake-time alertness patterns or it may be a pre-determined fixed value. If the further sleep time is below the second sleep threshold (block 704: yes), the alertness grades for the remaining time intervals in the awake-time alertness pattern are maintained in block 705. If the further sleep time is above the second sleep threshold (block 704: no), the alertness grades for at least one of the remaining time intervals in the awake-time alertness pattern are decreased in block 706. The decrease may be, for example, a constant decrease in the alertness grades or it may be proportional to how long time period there is be-tween the end of the further sleep event and the respective time interval. This reflects the adverse effects of the prolonged secondary sleep and resulting sleep inertia on the alertness for the remaining awake time. The process is then continued in block 707 to block 207 in FIG. 2.

Even a long secondary sleep may be beneficial for the user in terms of improving the alertness for the remaining awake-time period, for example when the user has sleep debt. An embodiment of the apparatus takes the sleep debt amount into account when evaluating the effect of the secondary sleep on the alertness grades. In this embodiment, the apparatus determines, on the basis of the sleep time and the target sleep time, a metric indicating an amount of sleep debt. If the metric indicates sleep debt above a sleep debt threshold and if the further sleep time is above the first sleep threshold, the apparatus may increase the alertness grades for the remaining time intervals in the awake-time alertness pattern determined. If the metric indicates sleep debt below the sleep debt threshold and if the further sleep time is above the first sleep threshold, the apparatus may maintain or decrease the alertness grades for the remaining time intervals in the awake-time alertness pattern determined. In this manner, the apparatus may manipulate the alertness pattern so that in case the long secondary sleep is used to reduce the sleep debt, it is considered to increase the alertness for the remaining awake time. On the other hand, if the long secondary sleep occurs in a case where there is no sleep debt or that the sleep debt is small (below the threshold), the long secondary sleep is considered not beneficial (maintain) and even disadvantageous (decrease) for the alertness of the remaining awake time.

In an embodiment, the sleep inertia is indicated as a separate indicator that is not incorporated into the alertness grades of the awake-time alertness pattern. In this embodiment, the sleep inertia indicator may be a function of the duration of the secondary sleep and, optionally, the sleep debt. The sleep inertia indicator may follow the above-described principles in the sense that it may indicate a low or non-existing sleep inertia, if the duration of the secondary sleep is below the first threshold, or even if the duration is above the first or the second threshold in a situation where the user had sleep debt before the secondary sleep. On the other hand, if the duration is above the first threshold, the sleep inertia indicator may indicate greater sleep inertia. If the duration is above the second threshold, the sleep inertia indicator may indicate even greater sleep inertia.

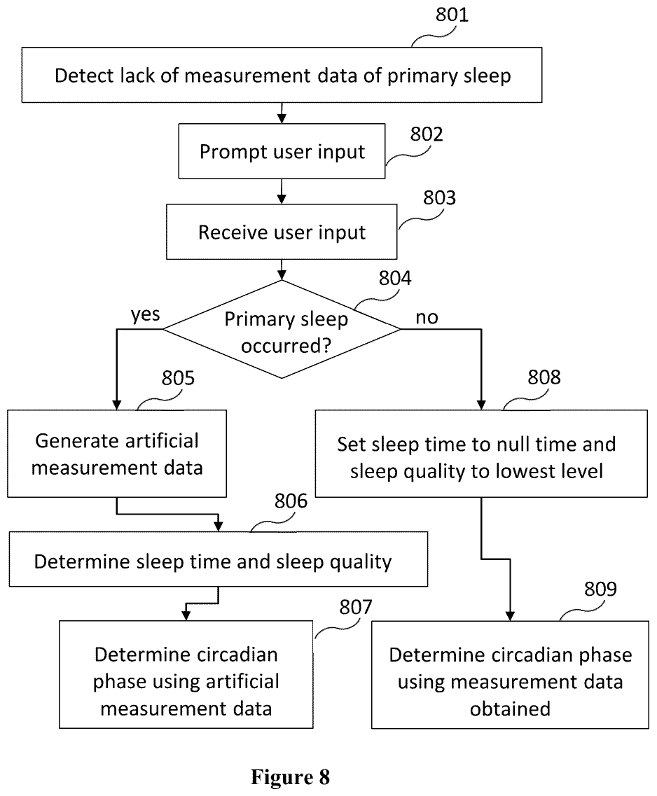

FIG. 8 illustrates an exemplary embodiment of the recursive alertness prediction procedure when a lack of measurement data is detected. According to the exemplary embodiment illustrated in FIG. 8 it is assumed that the plurality of time intervals occurs periodically based on a time period of 24 hours or a time period based on a circadian rhythm of the user, and there may be a gap in the measurement data, resulting in that artificial measurement data is generated. The gap may be caused by the user forgetting to wear the sensor device 104 during the primary sleep or a poor contact between the sensor device 104 and the user's skin. If the circadian rhythm measurements are based on the skin temperature data, the gap may be caused by the skin contact having been poor during the primary sleep. Generating the artificial measurement data enables obtaining more realistic alertness prediction results than by using the measurement data comprising the gap. However, there is a possibility that the gap results from that the user has not slept at all. It is important to distinguish the cause of the gap before estimating the awake-time alertness pattern, because the real alertness is fundamentally different in a case where the user has not had primary sleep and in a case where the user has had the primary sleep but has not worn the sensor device 104 or has worn the sensor device but with poor skin contact. The functionalities of FIG. 8 may be carried out within block 202 in FIG. 2.

Referring to FIG. 8, a lack of measurement data indicating a primary sleep event within a preceding time period of 24 hours, or a preceding time period based on the circadian rhythm is detected in block 801. Such a feature can be detected on the basis of lack of appropriate measurement signals from the sensors used for measuring the user. For example, the motion sensor may indicate that the user has not moved at all for hours, the heart activity sensor may receive only noise, and the skin temperature sensor may indicate temperature that is outside the nor-mal skin temperature range. The user is then prompted in block 802 via the user interface to input information on whether or not the primary sleep event has occurred. A user input comprising information on whether or not the primary sleep event has occurred is received in block 803 via the user interface. It is resolved in block 804 from the user input whether or not the primary sleep has occurred. If the primary sleep has occurred (block 804: yes), artificial measurement data on of an artificial primary sleep is generated in block 805. At least the sleep time and the sleep quality are determined in block 806 using the artificial measurement data generated. The artificial measurement data may be a copy of the measurement data of one of the earlier primary sleep events stored in the memory. The earlier primary sleep event may be a primary sleep event that reflects an average primary sleep of the user. The average primary sleep may be quantified in terms of an average heart rate during each of the primary sleep events stored in the memory. In other words, the average heart rate of the selected earlier primary sleep event may have the closest match with the average heart rate over all stored primary sleep events among the stored primary sleep events. Instead of the average heart rate during the primary sleep event, another metric may be used, e.g. average heart rate variability or average breathing rate. In another embodiment, the earlier primary sleep event is a primary sleep event of the same day of the week as the day from which the measurement data is missing, but from an earlier week. This is based on the assumption that the user has a substantially regular weekly rhythm. Yet another embodiment of selecting the earlier primary sleep event is based on an analysis of the measurement data of the primary sleep events by using machine learning and attempting to find regularities within the measurement data. This may include correlating the measurement data of the primary sleep events or other supervised or unsupervised machine learning principles. The machine learning may classify the primary sleep events on the basis of the measurement data, and the selection may then be made based on the classification.

The circadian phase of the user is determined in block 807 using the artificial measurement data generated. If the primary sleep has not occurred (block 804: no), the sleep time is set in block 808 to null time and the sleep quality is set to a lowest quality level. The circadian phase of the user is determined in block 809 using the measurement data obtained.

In an embodiment, upon generating the artificial sleep measurement for a primary sleep, the artificial measurement data is not used for the directly following awake-time alertness estimation but for the subsequent awake-time alertness pattern(s). This embodiment may be applied in a conservative embodiment where the artificial measurement data is considered less reliable, e.g. when the user has irregular sleeping habits. Since the sleep from the last night has the greatest effect on the alertness pattern, the algorithm may mandate that the measurement data from the previous night is real measurement data. Therefore, the generation of the awake-time alertness pattern for the awake-time directly following the primary sleep with lacking measurement data may be omitted.

As an alternative of using the artificial measurement data generated for estimating determining the circadian phase, the user may be prompted to enter the sleep time manually. Therefore, upon detecting the lack of measurement data indicating the primary sleep the apparatus may prompt the user to enter the sleep start time and sleep end time manually. Upon receiving the sleep start time and sleep as a user input, the circadian phase may be computed on the basis of the sleep start time and/or sleep end time. One option would be to estimate a sleep mid-point and compute the circadian phase from the sleep mid-point.

In an embodiment, the process of FIG. 2 or any one of its embodiments comprises computing a daily alertness grade for the user on the basis of the measurement data and/or the computed awake-time alertness pattern. The daily alertness grade may supplement the (hourly) alertness grades of the awake-time alertness pattern to provide the user with a numeric or verbal collation of the awake-time alertness. The daily alertness grade may be an average over the calculated alertness grades of the awake-time alertness pattern or over a time window after a wake-up time of a sleep event (primary or secondary sleep). The daily alertness grade may be calculated based on the primary sleep, secondary sleep or zero sleep. The daily alertness grade may comprise multiple verbal or numeric values representing different daily alertness levels, e.g. “good”, “fair”, and “modest”. The daily alertness grade may be used for detecting a lowered alertness level of the user and an alertness warning of the lowered alertness level may be given to the user via the user interface of the user device or the wearable device. For example, if the daily alertness grade is below a pre-determined daily alertness threshold, the warning is given to the user, and otherwise no warning is given.

The daily alertness grade may be split into a primary grade and a secondary grade. The primary grade may be based on the alertness grades calculated for the awake-time alertness pattern on the basis of the template and the measurement data from the primary sleep. There can be a single primary grade for one awake time (day) since it is based on the primary sleep event. Additional grade is based on alertness values calculated from secondary sleep. The secondary grade may consider the predicted alertness grades over a time window beginning after detection of the secondary sleep event (secondary sleep woke up time). The time window may thus be shorter than the time window for the primary alertness grade. There may be several secondary grades per day, depending on the number of secondary sleep events, and they can be displayed to the user separately. Similar to the primary grade, the secondary grade may be numeric and/or verbal. The purpose of the primary and secondary grade is to give the user a simple and comparable metric that represents the average daily alertness level.

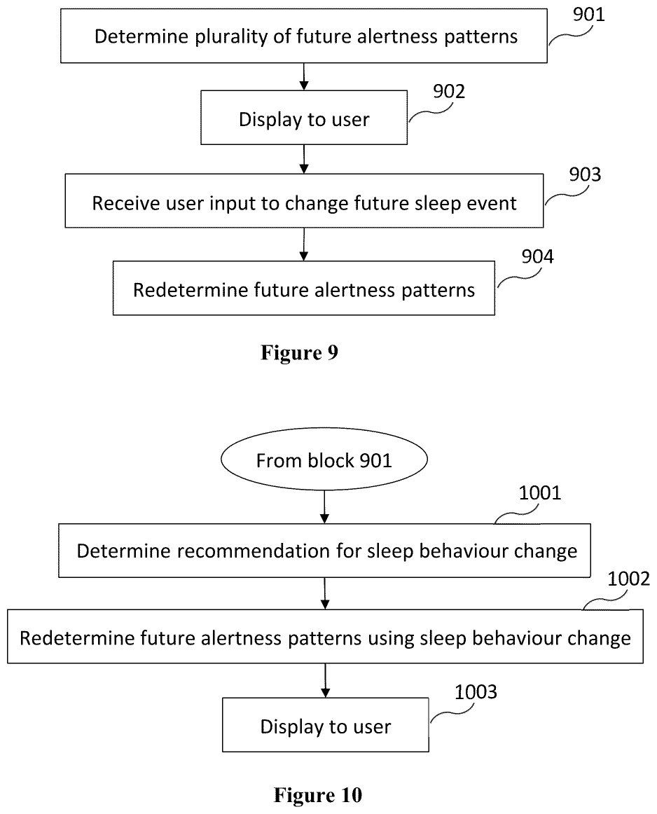

FIG. 9 illustrates an exemplary embodiment of using the recursive alertness prediction procedure for a plurality of future awake-time alertness patterns. Displaying the future alertness patterns can be used to indicate to the user how their sleeping behaviour affects alertness during awake-time.

Referring to FIG. 9, a plurality of future awake-time alertness patterns for future days is determined in block 901 by performing the recursive alertness prediction procedure per future awake-time alertness pattern, using the awake-time alertness pattern determined and the measurement data measured during one or more earlier primary sleep events of the user. The future awake-time alertness patterns may be computed by generating the artificial measurement data for the future (artificial) primary sleep events, on the basis of the measurement data of the earlier primary sleep events according to the above-described principles, and by estimating the future awake-time alertness patterns on the basis of the artificial measurement data. Displaying the plurality of future awake-time alertness patterns is caused in block 902 to the user via the user interface. This allows the user to see the evolvement of the awake-time alertness, if the user maintains his/her sleeping habits.

The process of FIG. 9 may also allow the user to modify the generated artificial measurement data and, in response to such a modification, the effects of the modified sleeping habits on the alertness may be simulated and presented to the user. A user input to change a future sleep time for at least one future primary sleep event between two consecutive future awake-time alertness patterns of the plurality of future awake-time alertness patterns determined is received in block 903. In response to the user input, at least one or the plurality of future awake-time alertness patterns following the at least one future primary sleep event with the future sleep time changed is redetermined in block 904 by performing the recursive alertness prediction procedure per future awake-time alertness pattern. Displaying the plurality of future awake-time alertness patterns redetermined may be caused to the user via the user interface. The parameters that the user may edit in block 903 include the sleep time and/or sleep quality of the primary sleep event(s), and/or adding secondary sleep event(s) and optionally editing the length of the secondary sleep events. In this manner, the user may try out various changes to the sleeping habits and see the effects of the changes on the awake-time alertness.

FIG. 10 illustrates an exemplary embodiment of using the recursive alertness prediction procedure to give recommendation to the user for changes in sleeping behaviour. The functionalities of FIG. 10 may be performed after block 901 in FIG. 9 and can be combined with the user-editable sleeping habits according to the embodiment of FIG. 9, or be an alternative to that.

Referring to FIG. 10, a recommendation for a change in sleeping behaviour of the user is determined in block 1001 using the measurement data measured during the one or more earlier primary sleep events of the user and the plurality of future awake-time patterns determined. The recommendation may be triggered upon detecting a degraded alertness of the user, e.g. the alertness grade being below a threshold level or the awake-time alertness pattern presenting lowered alertness levels (for example having no time interval with the highest alertness grade). The recommended change may be a change in the sleep time of the future primary sleep event or events, for example. Similar to the embodiment of FIG. 9, the alertness grades of the plurality of future awake-time patterns are redetermined in block 1002 by performing the recursive alertness prediction procedure per future awake-time alertness pattern, using the change in sleeping behaviour according to the recommendation. Displaying the recommendation for the change in sleeping behaviour and the redetermined alertness grades of the plurality of future awake-time alertness patterns is caused in block 1003 to the user via the user interface. In this manner, the user may be guided to improve the awake-time alertness of the coming days.

The embodiment of FIG. 10 may also be used to guide the user's sleeping habits so that the alertness level is high at a determined day in the future. For example, if the user is attending a competition on a certain day, the procedure of FIG. 10 may be used to provide the recommendations to the sleep in order to optimize the alertness during the competition.

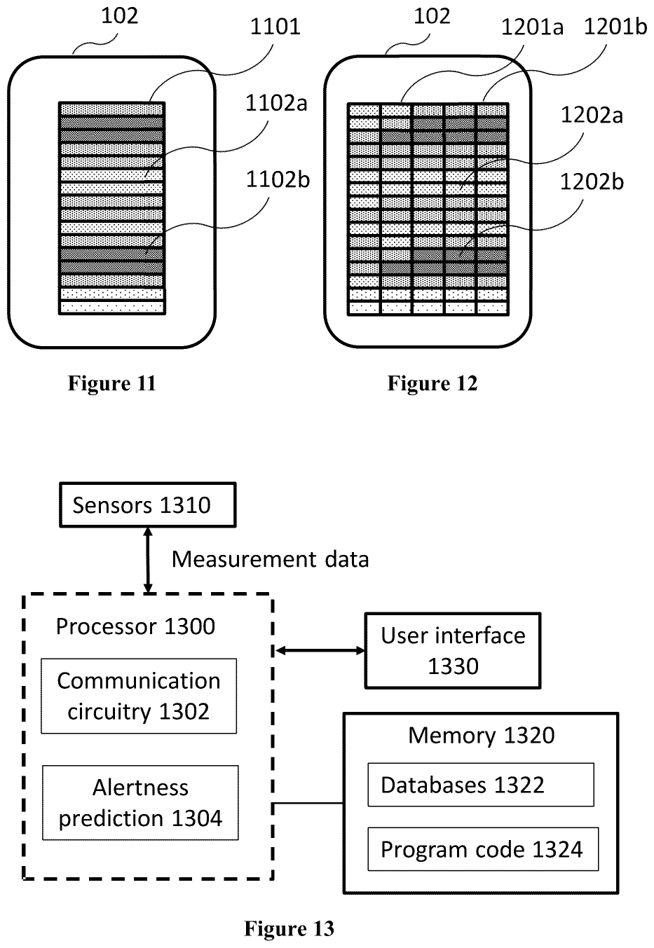

FIGS. 11 and 12 illustrate non-limiting exemplary display views of alertness patterns.

Referring to FIG. 11, a daily schedule 1101 of the user is illustrated on the display of the user device 102 as blocks of time intervals 1102a, 1102b. Each time interval may correspond to an hour, for example. The alertness grades for the time intervals are indicated by the denseness of the pattern of the blocks. Block 1102a presents a less dense pattern indicating a lower alertness grade for the respective time interval and block 1102b presents a denser pattern indicating a higher alertness grade for the respective time interval. However, this is just an example of indicating the alertness pattern, and numerous other display techniques may be utilised, such as colours. As illustrated in FIG. 11, a typical alertness pattern is bimodal, meaning that there are high-level alertness times in the morning and in the evening and an alertness dip between them. Accordingly, the default alertness pattern described above may have a bimodal structure. However, the bimodality may be affected by the sleep homeostasis and the circadian rhythm, as described above. For example, if the user has slept long enough and with high quality, the bimodality may not be so visible in the awake-time alertness pattern, and the high-level alertness may continue throughout the awake-time. The awake-time alertness pattern may still have the bimodal structure but the alertness grades of the time intervals may saturate to the highest alertness grade such that the degree of the bimodality decreases. As the sleep quality and/or sleep time degrades from their highest (most optimal) values, the degree of bimodality may increase. As the sleep quality and particularly the sleep time drops below a certain level, and/or the circadian phase shifts, the bimodality may again diminish in the awake-time alertness pattern. In such a case, the alertness grades of the time intervals may saturate to the lowest alertness grade(s) so that it indicates a substantially constant degraded alertness throughout the awake-time. According to these principles, the procedure of FIG. 2 may change the bimodality of the awake-time alertness pattern on the basis of the measured sleep time, sleep quality, and/or the circadian phase.

Referring to FIG. 12, a plurality of future daily schedules 1201a, 1201b of the user are illustrated on the display of the user device 102 as blocks of time intervals 1202a, 1202b. The alertness grades for the time intervals are indicated as explained above with FIG. 11 by the denseness of the pattern of the blocks.

However, the embodiments illustrated in FIGS. 11 and 12 are just examples indicating the alertness patterns, and numerous other display techniques may be utilised. The alertness grades in the awake-time alertness pattern may be provided in the form of score values within a determined range, e.g. 1 to 10, 0 to 10, or 0 to 100. The alertness grades may be provided in a verbal form, e.g. “High”, “Compromised”, and “Poor”, or “Good”, “Fair”, and “Modest”. The alertness grades may be provided in a coloured form, e.g. blue for high alertness, turquoise for compromised alertness, and green for poor alertness, or darker shades for high alertness and lighter shades for poor alertness.

The blocks and related functions described above in FIGS. 2 to 10 are in no absolute chronological order, and some of the blocks may be performed simultaneously or in an order differing from the given one. Other functions can also be executed between the blocks or within the blocks. Some of the blocks or part of the blocks can also be left out or replaced by a corresponding block or part of a block, for example, determining the sleep gate, determining the reliability metric value, or causing displaying to the user can be left out or replaced with each other.

The techniques described herein may be implemented by various means so that an apparatus implementing one or more functions/operations described above with an embodiment/example, for example by means of any of FIGS. 1 to 12 and any combination thereof, comprises not only prior art means, but also means for implementing the one or more functions/operations of a corresponding functionality described with an embodiment, for example by means of any of FIGS. 1 to 12 and any combination thereof, and it may comprise separate means for each separate function/operation, or means may be configured to perform two or more functions/operations. For example, one or more of the means for one or more functions/operations described above may be software and/or soft-ware-hardware and/or hardware and/or firmware components (recorded indelibly on a medium such as read-only-memory or embodied in hard-wired computer circuitry) or combinations thereof. Software codes may be stored in any suitable, processor/computer-readable data storage medium(s) or memory unit(s) or article(s) of manufacture and executed by one or more processors/computers, hardware (one or more apparatuses), firmware (one or more apparatuses), software (one or more modules), or combinations thereof. For a firmware or a software, implementation can be through modules (for example procedures, functions, and so on) that perform the functions described herein.

FIG. 13 is a simplified block diagram illustrating a structure of an apparatus (device, equipment) 1300 according to an embodiment and configured to perform at least some functionality described above for estimating alertness, for example by means of FIGS. 1 to 12 and any combination thereof. The apparatus may be applicable to or comprised in the user device. In other embodiments, the apparatus is applicable to or comprised in a sensor device, a wearable device, or a server computer. The apparatus may comprise at least one processor 1300 or processing circuitry and at least one memory 1320 including a computer program code 1324, wherein the at least one memory 1320 and the computer program code 1324 are configured, with the at least one processor 1300, to cause the apparatus to carry out the functions described above in connection with the processing circuitry. The processor 1300 may comprise a communication circuitry 1302 as a sub-circuitry configured to handle wireless connection with one or more sensor devices 1310 or internal connection between computer program modules through one or more application programming interfaces (APIs) in the apparatus. The sensor device(s) 1310 may be comprised in the apparatus, be external to the apparatus, or comprise both internal and external sensor devices. The sensor device(s) 1310 may comprise at least one of the following sensors: a heart activity sensor measuring the ECG, BCG, or PPG, a motion sensor or an inertial sensor measuring motion, an EEG sensor measuring the EEG, an EOG sensor measuring the EOG, a bioimpedance sensor measuring the bioimpedance or another galvanic property from a skin, and a respiratory rate sensor measuring the respiratory rate. The communication circuitry 1302 may be configured to receive measurement data form the sensor device(s) 1310. The communication circuitry 1302 may be configured to output alertness prediction patterns and/or other information through an API as described above.

The processor may comprise an alertness prediction module 1304 con-figured to determine the awake-time alertness pattern(s) according to any one of the embodiments of FIGS. 2 to 10 and any combination thereof. The alertness prediction module 1304 may be configured by the computer program code 1324 to map the obtained measurement data to the awake-time alertness pattern(s). The memory 1320 may store a database 1322 that provides rules for mapping of the obtained measurement data to the awake-time alertness pattern(s). When executing the processes according to any one of the above-described embodiment, the alertness prediction module 1304 may output the awake-time alertness pattern(s) to the user via one or more interface (IF) entities 1330, such as one or more user interfaces comprised in the apparatus or being external to the apparatus. The one or more interface entities 1330 are entities for receiving and transmitting information, such as communication interfaces comprising hardware and/or software for realising communication connectivity according to one or more communication protocols, or for realising data storing and fetching, or for providing user interaction via one or more user interfaces. The user interface may comprise a display screen or a display module for displaying the awake-time alertness pattern(s). The user interface may also comprise an input device for inputting information such as the target sleep or sleep occurrence. If the awake-time alertness pattern indicates that the user could change some sleeping habits to improve awake-time alertness, the alertness prediction module 1304 may determine a recommendation for changes in the sleeping habits, e.g. to increase sleep time and/or change falling asleep timing.

As used in this application, the term “circuitry” refers to all of the following: (a) hardware-only circuit implementations such as implementations in only analog and/or digital circuitry; (b) combinations of circuits and software and/or firmware, such as (as applicable): (i) a combination of processor(s) or processor cores; or (ii) portions of processor(s)/software including digital signal processor(s), software, and at least one memory that work together to cause an apparatus to perform specific functions; and (c) circuits, such as microprocessor(s) or a portion of a microprocessor(s), that require software or firmware for operation, even if the software or firmware is not physically present.

This definition of “circuitry” applies to all uses of this term in this application. As a further example, as used in this application, the term “circuitry” would also cover an implementation of merely a processor (or multiple processors) or portion of a processor, e.g. one core of a multi-core processor, and its (or their) accompanying software and/or firmware. The term “circuitry” would also cover, for example and if applicable to the particular element, a baseband integrated circuit, an application-specific integrated circuit (ASIC), and/or filed-programmable grid array (FPGA) circuit for the apparatus according to an embodiment.

The processes or methods described in FIGS. 2 to 12 and any combination thereof may also be carried out in the form of a computer process defined by a computer program. The computer program may be in source code form, object code form, or in some intermediate form, and it may be stored in some sort of carrier, which may be any entity or device capable of carrying the program. Such carriers include transitory and/or non-transitory computer media, e.g. a record medium, computer memory, read-only memory, electrical carrier signal, telecommunications signal, and software distribution package. Depending on the processing power needed, the computer program may be executed in a single electronic digital processing unit, or it may be distributed amongst a number of processing units.

It will be obvious to a person skilled in the art that, as the technology advances, the inventive concept can be implemented in various ways. The invention and its embodiments are not limited to the examples described above but may vary within the scope of the claims.

What is claimed is:

1. A method comprising:
performing a recursive alertness prediction procedure by performing operations comprising:
obtaining measurement data measured, from a user, by at least one sensor device, wherein the measurement data comprises data measured during at least a primary sleep event of the user;
determining, using the measurement data obtained, at least a sleep time, a sleep quality, and a circadian phase of the user;
selecting, in response to a first awake-time alertness pattern enabled for usage in the recursive alertness prediction procedure, the first awake-time alertness pattern as an alertness pattern template, wherein the first awake-time alertness pattern comprises a plurality of time intervals and an alertness grade per time interval;
selecting, otherwise, a default alertness model as the alertness pattern template, wherein the default alertness model comprises the plurality of time intervals and an alertness grade per time interval;
determining, using the sleep time, the sleep quality, the circadian phase determined, and the alertness pattern template selected, a second awake-time alertness pattern, to which the first awake-time alertness pattern is a preceding awake-time alertness pattern, by determining a plurality of alertness grades for the plurality of time intervals, an alertness grade per time interval; and
determining whether to enable or disable the second awake-time alertness pattern determined for a subsequent recursive alertness prediction procedure; and
causing displaying, to the user via a user interface, the awake-time alertness pattern determined.

2. The method according to claim 1, wherein the determining whether to enable or disable the second awake-time alertness pattern determined for the subsequent recursive alertness prediction procedure further comprises:
determining an amount of primary sleep measurement data comprised in the measurement data obtained;
enabling, in response to the amount of primary sleep measurement data detected being above a pre-determined data amount threshold, the second awake-time alertness pattern for the subsequent alertness prediction procedure; and
disabling, in response to the amount of primary sleep measurement data detected being below the pre-determined data amount threshold, the second awake-time alertness pattern for the subsequent alertness prediction procedure.

3. The method according to claim 1, wherein the performing the recursive alertness prediction procedure further comprises:
converting the measurement data from a local time of the user into a universal time frame;
determining the second awake-time alertness pattern in the universal time frame; and
converting the second awake-time alertness pattern determined to the local time of the user.

4. The method according to claim 1, further comprising:
obtaining measurement data measured, from the user, during at least one time interval of the second awake-time alertness pattern determined;
detecting a further sleep event of the user in the measurement data measured during the at least one time interval of the second awake-time alertness pattern determined;
determining, using the measurement data measured during the at least one time interval of the second awake-time alertness pattern determined, a further sleep time;
determining remaining time intervals in the second awake-time alertness pattern;
increasing, in response to the further sleep time being below a pre-determined first sleep threshold, the alertness grades for the remaining time intervals in the second awake-time alertness pattern determined; and
decreasing, in response to the further sleep time being above the pre-determined first sleep threshold, the alertness grade for at least one of the remaining time intervals in the second awake-time alertness pattern determined.

5. The method according to claim 4, further comprising:
maintaining, in response to the further sleep time being above the pre-determined first sleep threshold and below a pre-determined second sleep threshold, wherein the second sleep threshold is higher than the first sleep threshold, the alertness grades for the remaining time intervals in the second awake-time alertness pattern determined; and
decreasing, in response to the further sleep time being above the pre-determined second sleep threshold, the alertness grade for at least one of the remaining time intervals in the second awake-time alertness pattern determined.

6. The method according to claim 4, further comprising:
determining, on the basis of the further sleep time and a pre-determined target sleep time, a metric indicating an amount of sleep debt;
increasing, in response to the metric indicating sleep debt above a sleep debt threshold and if the further sleep time is above the first sleep threshold, the alertness grades for the remaining time intervals in the second awake-time alertness pattern determined; and
decreasing, in response to the metric indicating sleep debt below the sleep debt threshold and if the further sleep time is above the first sleep threshold, the alertness grades for the remaining time intervals in the second awake-time alertness pattern determined.

7. The method according to claim 1, wherein the performing the recursive alertness prediction procedure further comprises:
disabling, in response to the sleep time being shorter than a pre-determined target sleep time by at least a pre-determined sleep difference threshold, at least one alertness grade for the second awake-time alertness pattern and for the subsequent alertness prediction procedure, wherein the at least one alertness grade disabled comprises an alertness grade indicating the highest alertness in a range of alertness grades for the second awake-time alertness pattern.

8. The method according to claim 1, wherein the performing the recursive alertness prediction procedure further comprises:
storing, in the memory, as a plurality of circadian phases determined using the measurement data obtained, the plurality of circadian phases comprising the circadian phase;
determining, using the plurality of circadian phases stored, a phase shift in the circadian phase with respect to a preceding circadian phase;
determining a degree of the phase shift determined;
determining a decrease in alertness grades of at least a subset of the plurality of time intervals of the second awake-time alertness pattern, wherein the decrease is in a direct proportion with the degree of the phase shift, and wherein the subset of the plurality of time intervals comprises last adjacent time intervals of the second awake-time alertness pattern; and
disabling at least one alertness grade for the second awake-time alertness pattern for the subset of the plurality of time intervals, wherein the at least one alertness grade disabled comprises an alertness grade indicating a highest alertness for the subset of the plurality of time intervals of the second awake-time alertness pattern.

9. The method according to claim 1, wherein the plurality of time intervals occurs periodically based on a time period of 24 hours or a time period based on a circadian rhythm determined, the method further comprising:
detecting a lack of measurement data indicating a primary sleep event of the user within a preceding time period of 24 hours or a preceding time period based on the circadian rhythm determined;
prompting, in response to said detecting, the user, via the user interface, to input information on whether or not the primary sleep event of the user has occurred;
receiving, via the user interface, a user input comprising the information on whether or not the primary sleep event of the user has occurred;
generating, in response to the user input received indicating that the primary sleep event of the user has occurred, using measurement data measured during one or more earlier primary sleep events of the user, artificial measurement data of an artificial primary sleep event;
determining, in response to the user input received indicating that the primary sleep event of the user has occurred, using the artificial measurement data generated, the sleep time, the sleep quality, and the circadian phase of the user; and
determining, in response to the user input received indicating that the primary sleep event of the user has not occurred, using the measurement data obtained, the circadian phase of the user, and setting the sleep time to null time and the sleep quality to a lowest quality level.

10. The method according to claim 1, further comprising:
determining, using the second awake-time alertness pattern determined and the measurement data measured during one or more earlier primary sleep events of the user, a plurality of future awake-time alertness patterns for future days by performing the recursive alertness prediction procedure per a future awake-time alertness pattern;
causing displaying, to the user via the user interface, the plurality of future awake-time alertness patterns.

11. The method according to claim 10, further comprising:
receiving, via the user interface, a user input to change a future sleep time for at least one future primary sleep event between two consecutive future awake-time alertness patterns of the plurality of future awake-time alertness patterns determined; and
redetermining, in response to the user input, at least one of the plurality of future awake-time alertness patterns following the at least one future primary sleep event with the future sleep time changed by performing the recursive alertness prediction procedure per a future awake-time alertness pattern.

12. The method according to claim 10, further comprising:
determining, using the measurement data measured during one or more earlier primary sleep events of the user and the plurality of future awake-time alertness patterns determined, a recommendation for a change in sleeping behaviour of the user;
redetermining, using the change in sleeping behaviour according to the recommendation, the plurality of future awake-time alertness patterns by performing the recursive alertness prediction procedure per a future awake-time alertness pattern; and
causing displaying, to the user via the user interface, the recommendation for the change in sleeping behaviour of the user determined and the plurality of future awake-time alertness patterns.

13. The method according to claim 12, wherein the recommendation for the change in sleeping behaviour of the user comprises a longer sleep time and/or a more regular sleep time.

14. An apparatus comprising:
at least one processor; and
at least one memory including computer program code, the at least one memory and computer program code being configured to, with the at least one processor, cause the apparatus to perform a method comprising:
performing a recursive alertness prediction procedure by performing operations comprising:
obtaining measurement data measured, from a user, by at least one sensor device, wherein the measurement data comprises data measured during at least a primary sleep event of the user;
determining, using the measurement data obtained, at least a sleep time, a sleep quality, and a circadian phase of the user;
selecting, in response to a first awake-time alertness pattern enabled for usage in the recursive alertness prediction procedure, the first awake-time alertness pattern as an alertness pattern template, wherein the first awake-time alertness pattern comprises a plurality of time intervals and an alertness grade per time interval;
selecting, otherwise, a default alertness model as the alertness pattern template, wherein the default alertness model comprises the plurality of time intervals and an alertness grade per time interval;
determining, using the sleep time, the sleep quality, the circadian phase determined, and the alertness pattern template selected, a second awake-time alertness pattern, to which the first awake-time alertness pattern is a preceding awake-time alertness pattern, by determining a plurality of alertness grades for the plurality of time intervals, an alertness grade per time interval; and
determining whether to enable or disable the second awake-time alertness pattern determined for a subsequent recursive alertness prediction procedure; and
causing displaying, to the user via a user interface, the awake-time alertness pattern determined.

15. The apparatus according to claim 14, further comprising means for carrying out all steps of the method.

16. A computer program product comprising a computer-readable program code that, when executed by a computer system, causes execution of a method comprising:
performing a recursive alertness prediction procedure by performing operations comprising:
obtaining measurement data measured, from a user, by at least one sensor device, wherein the measurement data comprises data measured during at least a primary sleep event of the user;
determining, using the measurement data obtained, at least a sleep time, a sleep quality, and a circadian phase of the user;
selecting, in response to a first awake-time alertness pattern enabled for usage in the recursive alertness prediction procedure, the first awake-time alertness pattern as an alertness pattern template, wherein the first awake-time alertness pattern comprises a plurality of time intervals and an alertness grade per time interval;
selecting, otherwise, a default alertness model as the alertness pattern template, wherein the default alertness model comprises the plurality of time intervals and an alertness grade per time interval;
determining, using the sleep time, the sleep quality, the circadian phase determined, and the alertness pattern template selected, a second awake-time alertness pattern, to which the first awake-time alertness pattern is a preceding awake-time alertness pattern, by determining a plurality of alertness grades for the plurality of time intervals, an alertness grade per time interval; and
determining whether to enable or disable the second awake-time alertness pattern determined for a subsequent recursive alertness prediction procedure; and
causing displaying, to the user via a user interface, the awake-time alertness pattern determined.

17. The computer program product according to claim 16, wherein the computer program product is embodied on a computer-readable medium readable by the computer system.
